(19) United States
(12) Patent Application Publication
(10) Pub. No.: US 2026/0207477 A1
(43) Pub. Date: Jul. 23, 2026

(54) Microneedle Particles, Vehicles, and Substances of Interest

(71) Applicant: Georgia Tech Research Corporation, Atlanta, GA (US)

(72) Inventors: Mark R. Prausnitz, Atlanta, GA (US); Andrew Tadros, Atlanta, GA (US)

(21) Appl. No.: 19/143,568

(22) PCT Filed: Jan. 4, 2024
(86) PCT No.: PCT/US2024/010279

§ 371 Date: Jun. 26, 2025

Related U.S. Application Data
(60) Provisional application No. 63/436,984, filed on Jan. 4, 2023.

(51) Int. Cl. A61K 9/00; A61K 9/50

(52) U.S. Cl. CPC A61K 9/0021; A61K 9/50

(57) ABSTRACT

STAR particles, compositions, and methods of making the same, where the STAR particles and/or compositions are effective to prevent and/or reduce unwanted degradation or stabilizing and/or preserving STAR particles, substances of interest, and/or components of the STAR particles and/or the STAR particle-containing compositions.

### CROSS-REFERENCE TO RELATED APPLICATIONS

This application claims priority to U.S. Provisional Application No. 63/436,984, filed Jan. 4, 2023, which is incorporated herein by reference.

### BACKGROUND

Some embodiments of microneedle particles (i.e., STAR particles) are described in U.S. Pat. No. 11,219,816, which is incorporated herein by reference. STAR particles may provide effective mechanisms for improving delivery of bioactive compounds to biological tissues, such as the skin and other biological tissues with barrier or barrier-like properties. However, STAR particles, and/or such bioactive compounds therein, may be susceptible to unwanted degradation during fabrication, formulation, storage, application to the target tissue, and/or after their application to the target tissue. Degraded, or unstable, STAR particles may be less effective for their intended purpose. For example, degraded STAR particles may be less effective in mechanically disrupting a target tissue and/or delivering a substance of interest to the target tissue. It would be desirable to provide means for reducing or preventing unintended degradation of STAR particles and/or promoting the general stabilization of STAR particles and/or substances of interest contained therein.

### BRIEF SUMMARY

In one aspect, a packaged STAR particle composition is provided, the composition having a plurality of STAR particles which are configured to mechanically disrupt a biological tissue, a vehicle in which the plurality of STAR particles are dispersed, and a container holding the vehicle and STAR particles. The STAR particles, the vehicle, and the container are configured to maintain a substantially homogenous dispersion of the STAR particles within the vehicle and within the container. In some embodiments, the STAR particles and the vehicle have similar average densities. In some embodiments, the vehicle has a viscosity effective to prevent or limit dehomogenization of the STAR particles even when the average densities of the STAR particles and the vehicle are different from one another. In some embodiments, the vehicle includes at least one dispersant. In some embodiments, the container walls in contact with the vehicle have surface properties that inhibit adherence of the STAR particles thereto, optionally for example where the container walls have a coating effective to inhibit adherence of the STAR particles thereto. In some embodiments, the STAR particles possess electrostatic, steric hindrance, and/or magnetic properties that generate repulsive forces between the STAR particles, effective to prevent or limit agglomeration of the STAR particles. In some embodiments, the composition also includes at least one salt compound adapted to reduce the Debye length of a charge on the STAR particles. In some embodiments, the composition also incudes a surfactant adapted to inhibit interactions between hydrophobic or hydrophilic surfaces on the STAR particles.

In another aspect, a composition is provided, the composition having a plurality of STAR particles, and a vehicle in which the plurality of STAR particles are dispersed, where the vehicle has a viscosity of at least 100 cP, such that the viscosity of the vehicle is effective to at least partly maintain the dispersion of the STAR particles within the vehicle and/or limit dehomogenization.

In a further aspect, a composition is provided, the composition having a plurality of STAR particles having a structure formed at least partially of a first material, and a vehicle in which the plurality of STAR particles are dispersed, wherein the vehicle comprises a solvent for the first material, where the composition is configured such that the plurality of STAR particles are resistant to dissolution and/or unintended deactivation within the vehicle. In some embodiments, the STAR particles have a coating thereon which is substantially insoluble in the solvent of the vehicle. In some embodiments, the STAR particles are encapsulated in an encapsulation material which is substantially insoluble in the solvent of the vehicle. In some embodiments, the vehicle is saturated with solute effective to prevent or limit dissolution of the first material in the solvent of the vehicle. In some embodiments, the structure of the STAR particle is further formed of a second material which is substantially insoluble in the solvent of the vehicle.

In yet another aspect, a composition is provided, the composition having a plurality of STAR particles, and a vehicle in which the plurality of STAR particles are dispersed, where the STAR particles are adapted to degrade following application and use (i) by a selected change in pH, osmolarity, temperature, or ionic composition of the vehicle, or (ii) in response to an external stimulus. In some embodiments, the external stimulus may be exposure to atmospheric oxygen, light, or water.

In an even further embodiment, a packaged STAR particle composition is provided, the composition having a plurality of STAR particles which are configured to mechanically disrupt a biological tissue, a vehicle in which the plurality of STAR particles are dispersed, and a container comprising the vehicle and STAR particles, wherein the STAR particles and/or walls of the container in contact with the vehicle are coated with a cushioning material which is more deformable than the materials forming the STAR particles and/or the walls of the container.

In another aspect, a composition is provided, the composition having a plurality of STAR particles which are configured to mechanically disrupt a biological tissue, where the STAR particles having a surface coating composed of a material that is mechanically stronger than the underlying material(s) forming the STAR particles. In some embodiments, the underlying material(s) include an organic material and the surface coating material comprises an inorganic material. In some embodiments, the composition includes a substance of interest (SOI). In some embodiments, the SOI includes a bioactive agent. In some embodiments, the SOI is located in and/or on the STAR particles. In other embodiments, the SOI is located in the vehicle. In some embodiments, a method of administering a substance of interest (SOI) to a patient's skin is provided, the method of including applying the composition onto a skin surface, and manipulating the composition to cause the STAR particles to mechanically disrupt the skin surface.

In an additional aspect, a STAR particle composition is provided, the composition having a plurality of STAR particles which are configured to mechanically disrupt a biological tissue, a plurality of SOI particles separate from the plurality of STAR particles, and a liquid vehicle in which the plurality of STAR particles and the plurality of SOI particles are dispersed. In some embodiments, the STAR particles and/or the SOI particles have a coating thereon effective to substantially prevent dissolution of the STAR particles and/or the SOI particles in the liquid vehicle.

In another aspect, a STAR particle composition is provided, the composition having a plurality of STAR particles which are configured to mechanically disrupt a biological tissue, a first liquid phase, and a second liquid phase, where the plurality of STAR particles are dispersed in the first or second liquid phase, and a substance of interest (SOI) is provided in the first or second liquid phase. In some embodiments, the first liquid phase is a continuous phase and the second liquid phase is a discontinuous phase dispersed in the first liquid phase. In some embodiments, the STAR particles are dispersed in only one of the first liquid phase or the second liquid phase, and the SOI is dissolved in the other of the first liquid phase or the second liquid phase. In some embodiments, (i) the STAR particles are dispersed only in the first liquid phase, and the SOI is dissolved in the first liquid phase and the second liquid phase, (ii) the STAR particles are dispersed in the first liquid phase and the second liquid phase, and the SOI is dissolved only in the first liquid phase, (iii) the SOI is dissolved in the first liquid phase and the second liquid phase, and the STAR particles are dispersed only in the second liquid phase, (iv) the STAR particles are dispersed in the first liquid phase and the second liquid phase, and the SOI is dissolved only in the second liquid phase, (v) the STAR particles are dispersed only in the first liquid phase, and the SOI is dissolved only in the second liquid phase, (vi) the SOI is dissolved only in the first liquid phase, and the STAR particles are dispersed only in the second liquid phase, or (vii) the STAR particles are dispersed in the first liquid phase and the second liquid phase, and the SOI is dissolved in the first liquid phase and the second liquid phase.

### BRIEF DESCRIPTION OF THE FIGURES

The detailed description is set forth with respect to the accompanying drawings. The use of the same reference numerals may indicate similar or identical items. Various embodiments may utilize elements and/or components other than those illustrated in the drawings, and some elements and/or components may not be present in various embodiments. Elements and/or components in the figures are not necessarily drawn to scale.

FIG. IA is a plan view of a planar STAR particle according to one embodiment of the present disclosure.

FIG. 1B is a perspective view of the planar STAR particle of FIG. 1A.

FIG. 1C is a plan view of a planar STAR particle according to another embodiment of the present disclosure.

FIG. ID is a perspective view of the microneedle particle of FIG. 1C.

FIG. 1E is a side view of the microneedle particle of FIG. 1C.

FIG. 2 depicts agglomerated STAR particles, according to one embodiment of the present disclosure.

FIG. 3A depicts a reducing of a concentration of STAR particles to decrease a likelihood of STAR particle agglomeration, according to an embodiment of the present disclosure.

FIG. 3B depicts repulsive forces between some embodiments of STAR particles, to decrease a likelihood of STAR particle agglomeration according to another embodiment of the present disclosure.

FIG. 3C depicts STAR particles among a dispersant to decrease a likelihood of STAR particle agglomeration, according to another embodiment of the present disclosure.

FIG. 3D depicts weakly agglomerated STAR particles undergoing disruption of the agglomeration, according to another embodiment of the present disclosure.

FIG. 4A is a cross-sectional view of a planar STAR particle according to another embodiment of the present disclosure, in which the STAR particle has a coating.

FIG. 4B depicts a storage container, containing STAR particles, in which the storage container has a coating effective to retard STAR particle-container adhesion, according to one embodiment of the present disclosure.

FIG. 5A depicts insoluble STAR particles dispersed in a vehicle in a container, according to one embodiment of the present disclosure.

FIG. 5B depicts encapsulated STAR particles dispersed in a vehicle in a container, according to another embodiment of the present disclosure.

FIG. 6A depicts STAR particles settled at or near the bottom of a storage container.

FIG. 6B depicts STAR particles floating at or near the top of a vehicle within a storage container.

FIG. 6C depicts STAR particles adhered to sides of a storage container.

FIG. 7A depicts STAR particles stored in a dispersant, according to another embodiment of the present disclosure.

FIG. 7B depicts a coated storage container for a STAR particle-containing vehicle, according to another embodiment of the present disclosure.

### DETAILED DESCRIPTION

STAR particles and STAR particle-containing compositions are disclosed, along with methods of preventing and/or reducing unwanted degradation or stabilizing and/or preserving STAR particles, substances of interest, and/or components of the STAR particles and/or the STAR particle-containing compositions.

Stability of the STAR particles, and components of the STAR particle compositions, may be necessary to ensure the STAR particles can be produced, formulated, packaged, transported, stored and remain suitable for use as intended without unwanted degradation or loss of functionality.

In some cases, the goal is to stabilize the STAR particles during storage, and in other cases the goal is to stabilize the STAR particles during their application to tissue. The methods disclosed herein to stabilize may be the same for both goals, or the methods may be different for each goal. For example, it may be desirable to minimize or prevent interaction between STAR particles and the walls of a storage container, as well as among the STAR particles during storage. During application of the STAR particles to tissue, it may be desirable to minimize or prevent interaction between STAR particles and an applicator, i.e., the material or structure used to apply force to the STAR particles to cause them to interact with a tissue (e.g., a mechanical device, a gloved finger). One skilled in the art can adapt the concepts described herein for stabilizing in containers to stabilizing with applicators where appropriate.

As used herein, the phrase “packaged STAR particle composition” refers to a STAR particle composition disposed in a container suitable for storage and transport of the composition. The container may be essentially any rigid or flexible vessel known in the art that would be suitable for holding a quantity of the STAR particles, alone or in combination with a vehicle. It may, for example, be a vial, jar, pouch, bag or tube, and generally would include a cap, closure, plastic zip lock, or other means for sealing/closing, opening, and optionally reclosing, the vessel.

It may be important to segregate the STAR particles, the drug or other substance of interest (SOI), and other components (e.g., the vehicle) of the STAR particle formulations. In some embodiments, this may be achieved by having multiple phases present.

For example, one approach is to have a solid phase and a liquid phase. The solid phase is the STAR particle and the liquid phase is the vehicle. The formulation components are in the vehicle and/or in/on the STAR particle. The SOI may be in the vehicle and/or in/on the STAR particle. In some embodiments to segregate the solid phase from the liquid phase, the solid phase includes a coating that keeps the STAR particles from interacting with the vehicle (which could dissolve or swell or soften or otherwise adversely affect the STAR particle properties). For example, if the STAR particle is water-soluble and in an aqueous vehicle, the STAR particle could be coated with a water-insoluble material to keep the STAR particle from dissolving. The phase segregation could also keep the STAR particle from interacting with the SOI or other formulation components.

In some embodiments, there may be two solid phases. For example, the first solid phase may be the SOI, which is separate from the STAR particles (i.e., the second solid phase) such that there is a suspension of STAR particles and SOI particles in a liquid vehicle. There could be a coating around the SOI solid particles and/or the STAR particles, as needed to keep the STAR particles and the SOI particles from dissolving in the liquid vehicle. The SOI particles may have additional formulation components (e.g., excipients) in them.

In some embodiments, there may be two liquid phases. For example, one phase could be continuous and another phase discontinuous (e.g., emulsion). As another example, both phases could be continuous, but might become mixed prior to or during application to tissue (e.g., both phases become discontinuous). One phase could be aqueous and another phase non-aqueous. The STAR particles could be in one phase and not in the other phase. The SOI could be in the same phase as the STAR particles or in a different phase. For example, there could be water-soluble STAR particles in a non-aqueous phase and water-soluble SOI in an aqueous phase. In this way, a water soluble STAR particle (that is not dissolved) and a water soluble SOI (that is dissolved) can be in the same formulation. This is actually a three phase system because one phase is liquid, and the other phase is liquid with solid STAR particles suspended in the liquid.

The STAR particle-containing compositions and methods disclosed herein may enhance topical delivery of bioactive agents and other substances of interest to improve the desired effects of the compounds, to facilitate the compounds remaining in and/or on the target tissue, to facilitate extraction or removal of endogenous substances, compounds and/or specimens from a target tissue, and/or to promote systemic uptake of the compounds. The STAR particle-containing compositions and methods disclosed herein may be useful for diagnostic, prognostic, therapeutic, adjuvant, cosmetic and/or preventative purposes.

STAR particles may enhance topical administration of another substance or substances by mechanically disrupting the integrity of an outer/upper layer of skin (or other biological tissue) to facilitate the substance(s) local delivery into/onto the target tissue of a patient, and/or to promote its passage through a target tissue and uptake in the bloodstream for systemic delivery and/or lymphatics for systemic delivery, and/or to promote the substance(s) passage through a target tissue and uptake into another tissue or space including, but not limited to, joint spaces, tendons, ligaments, fascia, nerves, vessels, bones, muscles, glands, lymph nodes, subcutaneous tissue, adipose tissue, organs, and/or other tissues and spaces. The patient may be a human or other mammal or other animal or plant. The skin, or other biological tissue, may be in vivo or ex vivo.

In some embodiments, the STAR particles are configured to [1] at least partially disrupt a first type of biological tissue, and [2] prevent or decrease the likelihood that the STAR particles can disrupt a second type of off-target biological tissue. As used herein, the term “off-target tissue” refers to any tissue not intended to be disrupted by the STAR particles. For example, off-target tissues include, but are not limited to, the eye or ocular conjunctiva; oral, gastric, or vaginal mucosal membranes; and/or skin outside of the area of intended use when the targeted tissue is the skin. In particular, the second type of biological tissue, for example, may include the skin of the fingers, while the first type of biological tissue may include a tissue to be treated, for example, an area of the skin having a relatively thinner stratum corneum than the first type of biological tissue or a mucosal tissue. In this way, for example, the STAR particles may not disrupt, or may be less likely to disrupt, the skin of the fingers used to apply or rub the STAR particles onto/into the treatment area of the first biological tissue.

As used herein, a STAR particles being configured to “mechanically disrupt” a biologically tissue, particularly the stratum corneum of mammalian skin, particularly, human skin refers to the particle having dimensions and mechanical strength capable of creating holes or pores in the tissue surface. For example, the mechanically disrupting may be forming a penetration through the stratum corneum.

In some embodiments, the STAR particles disclosed herein may be configured, after its intended use, to partially or completely lose its mechanically disruptive properties so that the STAR particles cannot be reused.

### STAR Particles

The STAR particles include a core structure and one or more microneedle-like projections extending from the core structure. The microneedles may be structured to at least partially penetrate or otherwise mechanically disrupt a biological tissue, such as the stratum corneum of human skin (or other biological tissue). That is, the microneedles are dimensioned and possess the mechanical rigidity to enable them to be pressed into and penetrate the biological tissue, forming a microscale hole or channel therein. The microneedles may extend independently in any direction from the core structure.

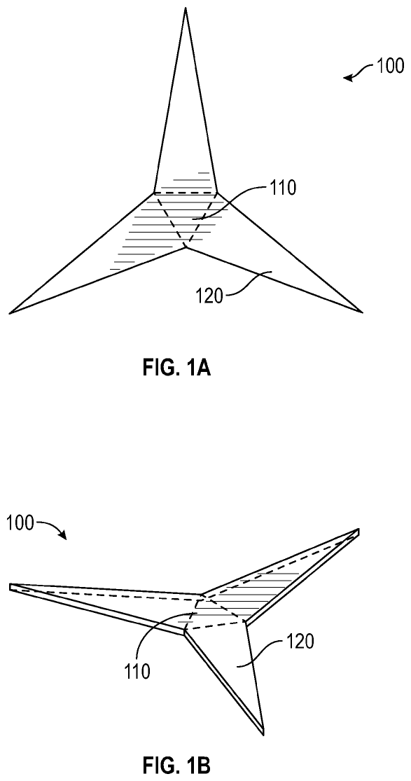

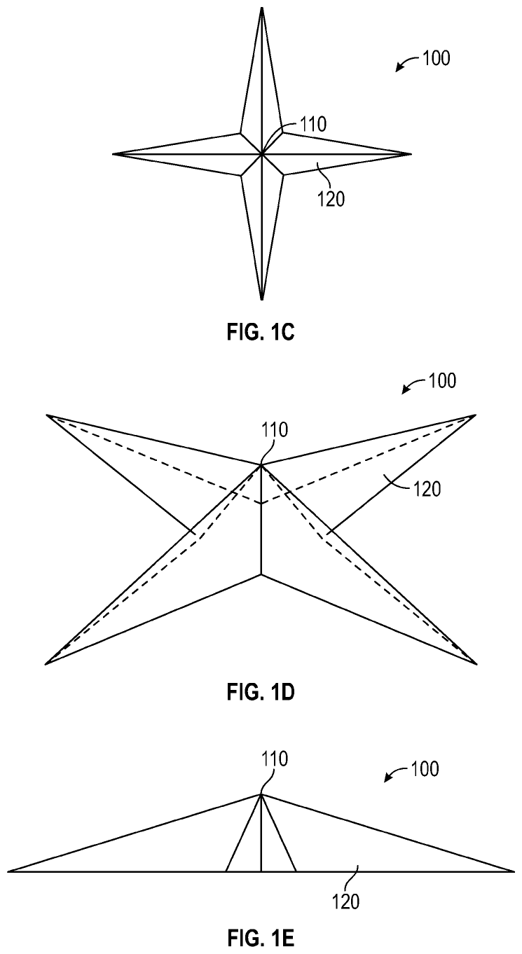

FIGS. 1A and 1B depict a STAR particle 100 according to a one embodiment. In this embodiment, the STAR particles 100 each have three microneedles 120 extending from the core structure 110 in the same plane, such that the STAR particle is referred to as a planar particle. The core structure typically is the portion of the microneedle particle that connects the microneedles, especially when there are multiple microneedles. The core structure may be a solid structure, a porous structure, or a hollow structure having one or more internal cavities. In other embodiments, the STAR particles may have two microneedles, four microneedles, five microneedles, six microneedles, seven microneedles, eight microneedles, nine microneedles, or ten microneedles extending from the core structure. In some embodiments, the microneedles extend from the core in different planes. For example, the STAR particle may have three, four, five, or more microneedles extending in different directions and planes, such that the STAR particle is referred to as a non-planar particle.

The microneedles of STAR particles may be tapered. In some embodiments, in the plan view, as shown in FIG. 1A, the microneedle 120 tapers from the core structure 110 to the tip end, but the height of the microneedles is substantially constant. In some other embodiments, the edges of the microneedle 120 may also be tapered, as the tapered edges are sharp and thereby able to penetrate stratum corneum more easily than untapered edges. For example, in some embodiments, as shown in FIGS. 1C-1E, the microneedles 120 may taper both in width and in height. That is, the height of the microneedle is largest at the core structure and smallest at the tip. In other variations, the core and a base portion of the microneedles may have a uniform height and only the distal tip portion of the microneedles is tapered. In variations, the taper may be from one or both sides of the STAR particle.

Various design features of the STAR particles may be selected to impart the particles with the functionality preventing the entire STAR particle from penetrating (i.e., mechanically disrupting) a biological tissue. These features may include the core structure itself, the microneedles themselves, or the spatial relationship between/among the microneedles or a subset of those microneedles. A combination of these features may be designed to prevent the entire STAR particle from penetrating a biological tissue.

For example, the core structure may have a size, shape, and/or a lack of sharp edges that permits one or more of the microneedles extending from the core structure to penetrate a biological tissue, but that inhibits all or substantially all of the core structure from penetrating into the biological tissue. As a further example, the microneedles may have a structural feature, such as tapering, that permits only a portion (i.e., the tip portion distal to the core structure) of the microneedles to penetrate a biological tissue. For example, a microneedle may have a shoulder or plateau that permits only the portion of the microneedle distal to the shoulder or plateau to penetrate the biological tissue. Such a configuration may prevent the core structure from contacting the biological tissue. In another example, the microneedle particle may have a generally curved shape that comes to a tip and, due to the geometry, only permits the tip portion to penetrate the biological tissue.

Generally, the microneedles of a STAR particle can have the same or different dimensions and/or geometries from one another. In one embodiment, the microneedles of a planar STAR particle have substantially the same dimensions and geometries.

The microneedles may have any shape effective to at least partially penetrate a biological tissue. In some embodiments, the microneedles are high-aspect-ratio structures having a length at least two times greater than its width at the base of the microneedle (i.e., at the interface of the microneedle and core structure). The length of a microneedle is the distance from the interface of the microneedle and the core structure's edge to the tip of the microneedle. In some embodiments, each of the microneedles independently has a length from 1 μm to 2,000 μm. In some embodiments, each of the microneedles independently has a length from 10 μm to 2,000 μm. In some embodiments, each of the microneedles independently has a length from 50 μm to 2,000 μm. In some embodiments, each of the microneedles independently has a length from 100 μm to 1,000 μm. In some embodiments, each of the microneedles independently has a length from 250 μm to 750 μm. In some embodiments, each of the microneedles independently has a length from 100 μm to 500 μm. In some embodiments, each of the microneedles has a length of about 350 μm.

In a particular embodiment, the STAR particles have three microneedles, wherein each of the microneedles independently has a length of about 1 μm to about 2,000 μm, about 10 μm to about 2,000 μm, about 50 μm to about 2,000 μm, about 100 μm to about 1,000 μm, or about 250 μm to about 750 μm. This STAR particle may be a planar particle.

The microneedles of the STAR particle may have a tip having a radius of curvature of about 0.1 μm to about 50 μm. In some embodiments, the microneedles have a tip having a radius of curvature of about 0.1 μm to about 50 μm, about 0.1 μm to about 25 μm, about 0.1 μm to about 20 μm, about 0.1 μm to about 15 μm, about 0.1 μm to about 10 μm, about 0.1 μm to about 5 μm, about 1 μm to about 10 μm, about 1 μm to about 7 μm, about 1 μm to about 5 μm, about 1 μm to about 4 μm, or about 1 μm to about 3 μm, about 5 μm to about 50 μm, about 5 μm to about 25 μm, about 5 μm to about 20 μm, about 5 μm to about 15 μm, or about 5 μm to about 10 μm. In some embodiments, each microneedle has a tip having a radius of curvature of about 5 μm to about 30 μm. The “tip” typically is the portion of the microneedles that first penetrates a biological tissue. In some embodiments, the STAR particles are shaped and sized to prevent, or reduce the likelihood of, the STAR particle becoming completely or irremovably embedded in the biological tissue. In some embodiments, the greatest dimension of the STAR particles is about 100 μm to about 5,000 μm, 100 μm to about 10,000 μm, about 250 μm to about 5,000 μm, about 500 to about 2,000 μm, or about 500 μm 1,000 μm. The “greatest dimension of the STAR particles” refers to the greatest of the following distances: [1] the distance between the tips of the two microneedles that are the farthest apart (if the microneedle particle includes two or more microneedles), or [2] the farther possible distance between a tip of a microneedle and the side of the core structure that is opposite the side from which the measured microneedle extends. A plurality of microneedle particles may include microneedle particles of one or more sizes.

In some embodiments, the microneedles of a STAR particle are planar microneedles. The phrase “planar microneedles,” as used herein, refers to two or more microneedles, each having either [1] a central axis that extends from the core structure in at least substantially the same plane, or [2] a tip that exists in substantially the same plane. The planar microneedles may include microneedles that extend from the core structure in the same direction, different directions, or a combination thereof. The planar microneedles also may include co-linear planar microneedles, which extend from opposite sides of the core structure in a manner that permits the central axis of each microneedle to at least substantially correspond with a single line. For example, where the STAR particles include two or more pairs of microneedles, the pairs of microneedles, but not necessarily all microneedles, may be co-linear.

When the microneedles are planar microneedles, the STAR particles may have a substantially planar, i.e., flat, structure. The substantially planar, i.e., flat, STAR particles may have a height (thickness) of about 1 μm to about 1,000 μm, about 5 μm to about 500 μm, about 10 μm to about 250 μm, about 50 μm to about 250 μm, about 50 μm to about 200 μm, about 50 μm to about 150 μm, about 75 μm to about 200 μm, about 75 μm to about 150 μm, about 75 μm to about 125 μm, or about 80 μm to about 120 μm. In some embodiments, the height of the microneedles is consistent throughout the length of the microneedle. That is, the height of the microneedle is the same where the microneedle contacts the core structure as at the tip. In some embodiments, the height of the microneedle particles decreases along the length of the microneedle. The height of the microneedle may be largest where the microneedle particle contacts the core structure and smallest at the tip.

In some preferred embodiments of tapered STAR particles, the height of the core in the center of the STAR particle is from 100 μm to 150 μm, and the radius of curvature at the tips of the microneedles of the STAR particle is from 5 μm to 30 μm.

The STAR particles may be made of one or more biocompatible materials, such as metals, polymers, biopolymers, ceramics, bioactive agents, sugars, sugar alcohols, or a combination thereof. The bioactive agents generally may include one or more drugs, one or more sensors, one or more cosmeceuticals, or a combination thereof. Therefore, the STAR particles may be made of a combination of bioactive components (drugs, small molecule excipients (e.g., trehalose), sensors, cosmeceuticals, or a combination thereof) and inactive components (metals, polymers, ceramics, sugars, etc.). If a portion of the STAR particle remains in and/or on a biological tissue after removal of the STAR particle, then the portion of the STAR particle remaining in and/or on the biological tissue may include at least one bioactive component, at least one inactive component, or a combination thereof.

In some embodiments, the STAR particles are made of water-insoluble material(s). In some embodiments, the STAR particles are made of, or include, at least one water-soluble and/or erodible material. When the STAR particles are made of water-soluble and/or erodible material(s), the STAR particles or a portion thereof may safely degrade if left in a biological tissue, or after disposal. In one example, the STAR particle has a matrix structure, which may consist of or include a water-soluble or bioerodible material. As used herein, the term “bioerodible” means that the structure/material degrades in vivo or ex vivo by dissolution, enzymatic degradation, hydrolysis, erosion, resorption, chemical reaction or a combination thereof. It would be understood that “ex vivo” refers in this case to STAR particles on the tissue surface, or otherwise present in the environment but not necessarily in contact with a biological tissue. Other methods of degradation of water-soluble and/or water insoluble STAR particles include, but are not limited to, dissolution, hydrolysis, degradation upon contact with sunlight (i.e., UV rays), or degradation resulting from a reaction with environmental factors (e.g., oxygen).

In some embodiments, the STAR particle is a metal microneedle particle. A metal microneedle particle is one in which all or substantially all of the structure of the microneedle particle is made of a metal or metal alloy (e.g., a stainless steel). In some other embodiments, a majority of the STAR particle is made of such metal or metal alloy materials.

In some embodiments, the STAR particle is a polymeric microneedle particle. A polymeric microneedle particle is one in which all or substantially all of the structure of the microneedle particle is made of one or more polymeric materials (e.g., biodegradable materials like poly(lactic-co-glycolic acid) (PLGA) or polycaprolactone (PCL) and/or water-soluble materials like carboxymethylcellulose or polyvinyl alcohol). In some other embodiments, a majority of the STAR particle is made of such one or more polymeric materials.

In some embodiments, the STAR particle is a ceramic microneedle particle. A ceramic microneedle particle is one in which all or substantially all of the structure of the microneedle particle is made of one or more ceramic materials (e.g., aluminum oxide, titanium dioxide, zinc oxide, iron oxides). In some other embodiments, a majority of the STAR particle is made of such one or more ceramic materials.

In some embodiments, all or substantially all of the structure of the microneedle particle is made of a bioactive agent and/or another other substance of interest. In some embodiments, a majority of the STAR particle is made of one or more drugs.

In some embodiments, the STAR particle is an excipient microneedle particle. An excipient microneedle particle is one in which all or substantially all of the structure of the microneedle particle is made of one or more pharmaceutically acceptable excipient materials known in the art (e.g., sugar, salt, starch, etc.).

In some embodiments, the STAR particle has a structure that is formed of a combination of (i) at least one metal (or metal alloy), (ii) at least one polymeric material, (iii) at least one ceramic material, and/or (iv) a at least one bioactive component.

The STAR particles provided herein may be made by any suitable method capable of forming a desired geometric shape of the STAR particles. Non-limiting examples of such methods include molding, mechanical or chemical etching, laser cutting, 3D printing, or other microfabrication techniques known in the art. For example, the STAR particles may be formed by laser etching a sheet of a material. As a further example, the STAR particles may be made using a molding process that may include placing a material of construction in a mold having cavities that correspond to the desired geometry of the resulting STAR particles. The material of construction may be a polymer or precursor thereof, and may be loaded into the mold in a powder or liquid form (e.g., molten polymer and/or polymer dissolved or dispersed in a vehicle), and then solidified into solid monolithic form in the mold. In another example, an array of discrete particles is formed from a solid sheet of the material by a process that includes at least one of etching, punching, or cutting, such as laser cutting. The STAR particles also may be sintered, densified, and/or mechanically hardened via heating, cooling, chemical modification, light exposure, drying, compressing, and/or other processes.

### Star Particle-Containing Compositions

In various embodiments, the STAR particles are provided as a composition that facilitates application of the STAR particles to a target tissue site, e.g., a biological tissue surface, such as mammalian skin. For example, the composition may include or consist of STAR particles dispersed in a suitable medium that can flow. The medium may be a liquid, solution, lotion, cream, ointment, gel, paste, emulsion, aerosol foam or spray, powder, or semi-solid. The suitable medium is referred to herein as a “vehicle”.

Essentially any suitable biocompatible vehicle may be used in the STAR particle-containing composition. The vehicle may be an aqueous medium and/or a non-aqueous medium. The vehicle may include water, stabilizers, pH modifiers, thickening agents, or other pharmaceutically acceptable excipients known in the art for use in topical therapeutic applications, including materials that listed as Generally Recognized as Safe (GRAS) by the U.S. Food and Drug Administration.

The STAR particle-containing composition may include one or more bioactive agents (e.g., a therapeutic or prophylactic agent) and/or other substances of interest (e.g., diagnostic agents, sensors, cosmetics/cosmeceuticals). The bioactive agent, and/or the other substance of interest, may be disposed in or on the STAR particles, in the vehicle, or both in or on the STAR particles and in the vehicle. In some embodiments, the bioactive agent is dissolved in the vehicle. In some embodiments, the bioactive agent is dispersed, i.e., as a particulate suspension, in the vehicle.

The STAR particle-containing composition generally has a viscosity suitable for its intended storage, packaging, and use (e.g., application to a target tissue). In some embodiments, the STAR particle-containing composition is a viscous composition, such as wherein the vehicle of the composition has a viscosity of at least 100 cP. In some embodiments, the composition, or the vehicle, has a viscosity of about 1,000 cP to about 200,000 cP, about 1,000 cP to about 150,000 cP, about 1,000 cP to about 100,000 cP, about 1,000 cP to about 75,000 cP, or about 1,000 cP to about 50,000 cP. In some embodiments, the STAR particle-containing composition is a non-viscous composition, having a viscosity of less than 100 cP, for example, about 5 cP to about 75 cP, about 5 cP to about 50 cP, or about 5 cP to about 25 cP. In some embodiments, the STAR particle-containing composition has a viscosity of about 1 cP.

The concentration of STAR particles in the vehicle may be selected based on the particular application, but generally would be selected to achieve the intended function of the STAR particles at a particular tissue site. For example, the STAR particle concentration may be selected to be sufficient to create enough holes in the stratum corneum to deliver a therapeutically effective amount of a bioactive agent into the skin at the site of application of the STAR particle-containing composition.

In some embodiments, the concentration of STAR particles in the vehicle ranges from about 100 to about 100,000 particles per cm³ of the vehicle. In some embodiments, the concentration of STAR particles in the vehicle ranges from about 500 to about 50,000 particles per cm3 of the vehicle. In some embodiments, the concentration of STAR particles in the vehicle ranges from about 1,000 to about 25,000 particles per cm³ of the vehicle. In some embodiments, the concentration of STAR particles in the vehicle is greater than 10,000 particles per cm³ of the vehicle. In some embodiments, the concentration of STAR particles in the vehicle is less than 10,000 particles per cm³ of the vehicle.

In some embodiments, the concentration of STAR particles in the vehicle ranges from about 0.1 wt % to about 30 wt % of the vehicle. In some embodiments, the concentration of STAR particles in the vehicle ranges from about 1 wt % to about 20 wt % of the vehicle. In some embodiments, the concentration of STAR particles in the vehicle ranges from about 5 wt % to about 15 wt % of the vehicle. In some embodiments, the concentration of STAR particles in the vehicle ranges from about 8 wt % to about 12 wt % of the vehicle. In some preferred embodiments, the concentration of STAR particles in the vehicle is about 5 wt % to about 10 wt % of the vehicle.

STAR particle compositions may also include at least one substance of interest. As used herein, “substance of interest” refers to a molecule or collection of matter that has a prophylactic, therapeutic, diagnostic, or cosmetic purpose. Substances of interest may include, but are not limited to, active pharmaceutical ingredients, vaccines, allergens, vitamins, cosmetic agents, cosmeceuticals, diagnostic agents, sensors, markers (e.g., colored dyes or radiological dyes or markers), other bioactive agents, and other materials that are desirable to introduce into or onto a biological tissue. A list of substances of interest is included in U.S. Pat. No. 11,291,816, which is incorporated herein by reference.

The substance of interest may be a small molecule, polymer, peptide, or biologic agent. In some embodiments, the substance of interest is a biological agent or a living organism. In further embodiments, the substance of interest has electronic properties. For example, the substance of interest may be responsive to radio-frequency identification (RFID).

### Methods of Stabilizing Star Particles and Star Particle-Containing Compositions

STAR particles, and STAR particle-containing compositions, may be susceptible to degradation during fabrication, formulation, packaging, transportation, storage, when in use and/or after use. As used herein, “degradation” refers to a change in the STAR particles, vehicle, substance of interest, or any combination thereof from a functional and safe state to a (i) less functional or non-functional state and/or (ii) a less safe or an unsafe state. Here, a functional state refers to a state in which the STAR particles can perform a certain intended function. A less functional or non-functional state refers to loss of the ability to perform that intended function, but does not mean that the STAR particles are unable to perform any other function.

In some embodiments, the STAR particles, vehicle, and/or substance of interest may degrade and/or lose functionality as the result of an external stimulus. As used herein, “external stimulus” refers to any condition applied to the STAR particles, vehicle, and/or substance of interest that results in a change to the functionality, usability, and/or safety of the STAR particles, vehicle and/or substance of interest. These may include, but are not limited to, (a) exposure to visible light, (b) changes in temperature, (c) changes in pressure, (d) addition, modification, or removal of chemical entities, (e) application of ultrasound, (f) application of electromagnetic radiation (e.g., ultraviolet, visible, infrared radiation), (g) application of electrical and/or magnetic fields, (h) exposure to the atmosphere conditions (e.g., oxygen), (i) application of mechanical forces (e.g., compression, tension, shearing), (j) exposure to a target tissue, and others.

In other embodiments, the STAR particles, vehicle, and/or substance of interest may degrade as the result of a chemical reaction of one or more components of these materials. As used herein, “chemical reaction” refers to the formation and/or breaking of non-covalent and/or covalent bonds.

The STAR particles may be adapted to degrade following their application and use. The term “degrade” in that context means loss of their ability to mechanically disrupt a biological tissue. Such a functional loss may be caused by one or more material(s) of construction of the STAR particles undergoing a phase change and/or breaking apart such that the STAR particle no longer has the mechanical strength or structural dimensions needed to mechanically disrupt a biological tissue, e.g., to form a hole in the stratum corneum. Such degradation may be caused (i) by a selected change in pH, osmolarity, temperature, or ionic composition of the vehicle, and/or (ii) in response to an external stimulus.

As described herein, the STAR particles, vehicles, and/or substances of interest may be stabilized to reduce the likelihood of, and/or materially delay, their degradation. As used herein, “stabilization” refers to the complete or partial inhibition of the processes of degradation to maintain functionality of the STAR particles, vehicles, and/or substances of interest. Stabilization may be achieved by the addition of stabilizing compounds, presence of certain inherent or external conditions, and/or application of external stimulus to the STAR particles, vehicle, and/or substance of interest.

Degradation of the STAR particles may lead to premature loss of functionality, i.e., unintended deactivation. As used herein, the phrase “unintended deactivation” in reference to STAR particles refers to the STAR particles'loss of ability to mechanical disrupt, or puncture, the stratum corneum (or other tissue) prior to its intended use.

In some embodiments, only the vehicle and/or the substance of interest may be susceptible to degradation as a result of physical and/or chemical changes. An exhaustive list of physical and chemical changes that may cause destabilization can be found in “Drug stability for pharmaceutical scientists” (Thorsteinn Loftsson, Academic Press 2014). In one embodiment, instability at the molecular level may destabilize the vehicle and/or substance of interest. Molecular instabilities are described in “Overview of pharmaceutical excipients used in tablets and capsules” (R H Dave, Drug Topics 2008). In another embodiment, instability causing degradation occurs at a non-molecular level, examples of which described in “Pharmaceutical Suspensions—From Vehicle Development to Manufacturing” (A K Kulshreshtha et al., Springer 2010).

Accordingly, the vehicles and/or substances of interest composition may be formulated to include antioxidants, buffers, emulsifiers, sugars, carbohydrates, and/or other agents known in the art to be effective for physically and/or chemically stabilizing pharmaceutical ingredients that may be included in the STAR particles or in the vehicle of the STAR particle-containing compositions. For example, a listing of stabilizers and stabilization methods can be found in “Pharmaceutical Dosage Forms and Drug Delivery Systems” (L V Allen, Wolters Kluwer Health 10^{th} Ed.), “Drug Stability for Pharmaceutical Scientists” (T. Loftsson, Academic Press, 1^{st} Ed), and “The development of microgels/nanogels for drug delivery applications” (J K Oh, Progress in Polymer Science (2008) 33(4) pp. 448-477), which are incorporated herein by reference.

In some embodiments, the STAR particle-containing composition, a vehicle, and a substance of interest, may destabilize. For example, the substance of interest may prematurely diffuse out of the STAR particles and into the vehicle. In another example, the substance of interest may degrade within the STAR particles and/or within the formulation. In still another example, a substance of interest designed to be dispersed within the vehicle may undesirably be absorbed by the STAR particles and/or by the storage container, resulting in destabilization of the STAR particles, loss of efficacy of the formulation, and/or the substance of interest. In still another example, a substance of interest designed to be coated onto and/or into the STAR particle may prematurely dissolve and/or leach off of the STAR particle prior to its intended use.

In some embodiments, the STAR particles may destabilize during storage. For example, the STAR particles may agglomerate, weaken, break, become deformed, chemically destabilize, swell, change in size, change in shape, change in hardness, change in porosity, and/or de-homogenize within the vehicle. Such destabilizations may be controlled as described below.

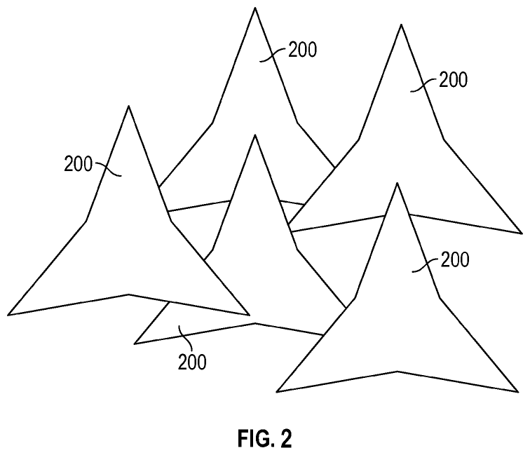

In some embodiments, the vehicle or STAR particles may be inherently susceptible to agglomeration. FIG. 2 depicts a plurality of agglomerated STAR particles 200. The vehicle containing the STAR particles may dry out, increasing the concentration of STAR particles within the vehicle. This increased concentration results in closer spatial proximity of the STAR particles within the vehicle and thereby may increase the likelihood of interactions between and agglomeration of the STAR particles. The STAR particles may also have magnetic dipoles and/or electrostatic charge, where the presence of these dipoles and/or charges increases the likelihood of interaction between the STAR particles. The ionic concentration within the vehicle may also cause changes to the surface charge of the STAR particles, increasing the likelihood of agglomeration. The STAR particles may also have chemical modifications to their surface that cause them to have adhesive properties which may cause them to further agglomerate when they come in close proximity to each other.

In some embodiments, agglomeration may be triggered by a chemical reaction. In some embodiments, the chemical reaction is a reaction of at least one reagent contained within the vehicle. Alternatively, the reagent is not contained within the vehicle, but rather is added by a user when applying the STAR particles. In some embodiments, the chemical reaction is a reaction of at least one encapsulated reagent contained within the vehicle. The encapsulated reagent may react with either an encapsulated or a non-encapsulated reagent.

An external stimulus may be applied, intentionally or inadvertently, to a STAR particle-containing composition, where the external stimulus causes agglomeration. In some embodiments, the force of applying a STAR particles-containing composition onto a tissue surface may cause agglomeration or may deform the STAR particles in a manner (e.g., where the tips bend and form hook-like structures that can mechanically link with other STAR particles) that increases the likelihood of their agglomeration. In some embodiments, the STAR particles 300 may interact with the physical and/or chemical properties of the target tissue, increasing the likelihood of or causing agglomeration.

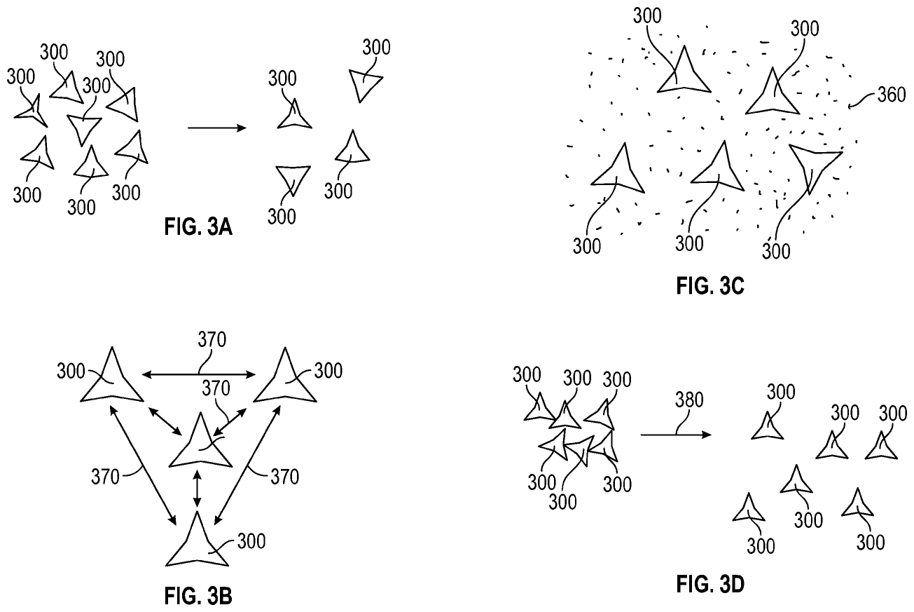

FIGS. 3A-3D depict methods of preventing and/or disrupting agglomeration. In general, agglomeration may be prevented by minimizing the chance and/or strength of STAR particles 300 interacting with other STAR particles 300, with a container in which the STAR particles 300 are stored, and/or with materials or compositions within the vehicle in which the STAR particles 300 are dispersed.

In some embodiments, as shown in FIGS. 3A-3C, reducing the likelihood of interactions between STAR particles 300 may be effective to reduce and/or prevent agglomeration. For example, as illustrated in FIG. 3A, reducing the concentration of STAR particles 300 within the vehicle (i.e., increasing the average spacing among the particles) may be effective to reduce the likelihood of interactions between STAR particles 300. Where STAR particles 300 are less likely to contact one another, agglomeration is less likely to occur.

FIG. 3B depicts an embodiment in which repulsive forces 370 between the STAR particles 300, which are effective to maintain a relatively homogeneous distribution of the STAR particles within the vehicle. In some embodiments, the repulsive force 370 is an electrostatic force. In some embodiments, the repulsive force is a magnetic force. In some embodiments, the repulsive force is the result of steric hindrance. In some embodiments, STAR particles may be at least partially or fully encapsulated in a material that limits the ability of STAR particles to agglomerate and/or interact through macroscopic, microscopic, and/or atomic steric hindrance.

The material used to encapsulate the STAR particle may be composed of metals, polymers, biopolymers, ceramics, bioactive agents, sugars, sugar alcohols, other materials that are not soluble in the vehicle or a combination thereof. The material used to encapsulate the STAR particle may subsequently undergo degradation if STAR particle agglomeration is desired.

In some embodiments, the STAR particles may also be lubricated to prevent interactions between STAR particles that may cause agglomeration. In some embodiments, at least one compound in the vehicle lubricates the STAR particles. In some embodiments, the STAR particles are coated with and/or encapsulated in a lubricant. In some embodiments, the surface energy of the STAR particles functions as a lubricant, preventing interactions between the STAR particles.

The vehicle in which the STAR particles are dispersed may be effective to prevent agglomeration. In some embodiments, the viscosity of the vehicle is effective to prevent the STAR particles from agglomerating. That is, the viscosity of the vehicle is high enough so that movement of the STAR particles within the vehicle is limited. In some embodiments, as shown in FIG. 3C, the vehicle includes a dispersant 360 that may reduce interactions between the STAR particles 300 and prevent agglomeration. As used herein, a “dispersant” or dispersing agent” is a substance, typically a surfactant, that is added to a suspension of solid or liquid particles in a liquid (such as a colloid or emulsion) to improve the separation of the particles and to prevent their settling or clumping. A variety of suitable dispersants known in the art may be used with the STAR particles, Pharmaceutically acceptable dispersants are known in the art include, but are not limited to, acacia, tragacanth, bentonite, carbomer, cellulose, dextrin, maltodextrin, gelatin, methylcellulose, carboxymethyl cellulose, polyvinyl alcohol, hyaluronic acid, pectin, and/or starch. Other agents generally known in the art may be found in the official monographs in the USP. In some embodiments, minimizing turbulence and/or maintaining laminar flow within the vehicle may decrease the likelihood of movement of the STAR particles. Where the STAR particles are less likely to move, agglomeration may be reduced or eliminated.

In some embodiments, as illustrated in FIG. 3D, the STAR particles 300 may be weakly agglomerated (shown on left side of arrow). Weakly agglomerated STAR particles 300 may be disrupted following application of an external stimulus 380 (shown in right side of arrow). For example, a storage container, containing STAR particles and a vehicle, may be manually shaken or shaken with the aid of suitable mixing or vibration equipment known in the art, to break up any agglomerates that have developed.

Some STAR particles may be inherently mechanically fragile or may weaken over time, for example as a result of exposure to another material, such as the vehicle, the substance of interest, or any other component present within the vehicle or storage container. Such STAR particles may break upon collision with other STAR particles and/or with the walls of a storage container in which the STAR particle-containing composition in disposed. In some embodiments, fluid flow within the vehicle may also cause the STAR particles to break. In some embodiments, contact with other STAR particles or the storage container, or fluid flow in the vehicle, can cause STAR particles that have not been weakened to break.

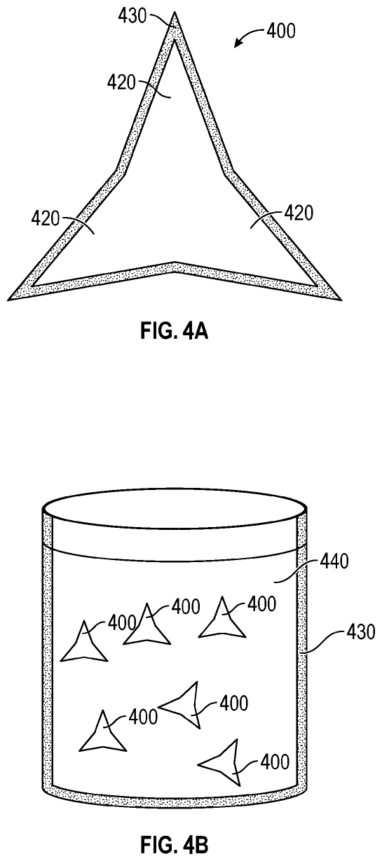

In some embodiments, as shown in FIG. 4A, the STAR particle 400 may have a coating 430 that prevents weakening and/or breaking of the microneedles 420 or otherwise improve the mechanical strength of the STAR particle 400. For example, where the STAR particles are susceptible to being weakened by an aqueous solution, the coating may be hydrophobic, and vice versa. The coating may also serve as a barrier to prevent fluid from contacting the STAR particles. The coating may also be composed of a mechanically strong but brittle sacrificial material that breaks, disintegrates, and/or cracks when it comes in contact with another material, such as another STAR particle or the wall of the storage vessel, and thereby serves as a protective layer for the STAR particle. As used herein, the phrase “mechanically stronger” as used to compare a material coating a STAR particle with the underlying material(s) forming the STAR particle means that coating material is better able than the underlying structural material to withstand an applied load without failure or plastic deformation. For example, the coating material may have a greater compressive strength than the underlying structural material.

In some embodiments, the storage container for the STAR particles may be configured to retard or prevent breakage resulting from contact with the walls of the storage container. For example, in one embodiment as shown in FIG. 4B, the walls of the storage container are coated with a coating 430 to retard contact and/or adhesion between the container and the STAR particles 400. For example, the coating may be a lubricant, may have an electrical charge that repels the STAR particles, or may be a relatively softer material than the container and/or the STAR particle material of construction. The repulsive properties may be inherent or may be enhanced by the presence of a coating. Exemplary coatings may include, but are not limited to, a polymer (e.g., polyvinyl alcohol, poly(ethylenimine), poly(methyl methacrylate),), biopolymers (e.g., deoxynucleic acid, ribonucleic acid, poly amino acids such as poly(l-lysine), chitosan), proteins, ceramics (metal oxides), metals, sugars and/or other excipients generally known in the art.

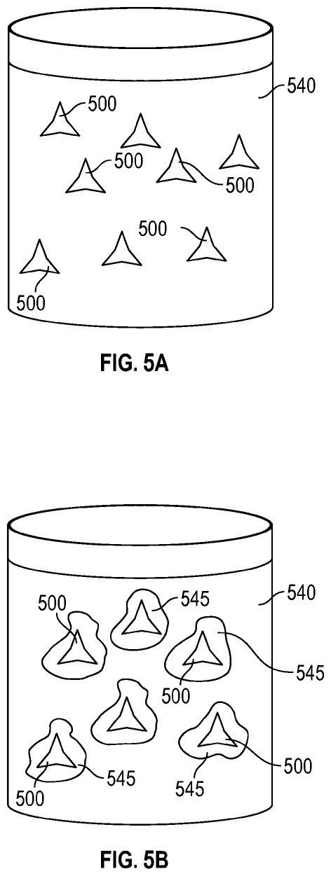

The materials forming the STAR particles, or at least an exterior portion thereof, and the vehicle may be selected or formulated to prevent degradation of the STAR particles in the vehicle. For example, as shown in FIG. 5A, the STAR particles 500 are insoluble in the vehicle 540. In some cases, for instance, the STAR particles are formed of one or more materials that are substantially insoluble in water, and the vehicle is an aqueous vehicle. In some other cases, for instance, the STAR particles are formed of one or more water soluble materials, and the vehicle is a non-aqueous vehicle. In some other cases, the STAR particles may be formed of a material (such as a water-soluble material) which is then coated with a coating material that is water insoluble, and the vehicle is an aqueous vehicle, wherein the coating materials acts as a barrier to prevent dissolution of the water-soluble material of the STAR particles dispersed in the aqueous vehicle. In a similar variation, as shown in FIG. 5B, the STAR particles 500 are encapsulated in an encapsulation material 545 which is insoluble in the vehicle 540.

In some preferred embodiments, the STAR particles are made of one or more water soluble materials but have a coating thereon which is substantially water insoluble, so the STAR particles will not dissolve in storage or during use, wherein the coating is configured to dissolve later and thereby deactivate the (used) STAR particles. For example, the coating could be made of a material that is water-insoluble at low pH but is water-soluble at neutral pH. Such materials are known in the art, for example Eudragit™ polymers.

In some embodiments, the vehicle is a non-solvent for the STAR particle material of construction. In some other embodiments, the vehicle is a solvent for the STAR particle material, but the vehicle is saturated or supersaturated with dissolved material (solute), such that no additional material (i.e., no material from the STAR particle) can dissolve into the vehicle.

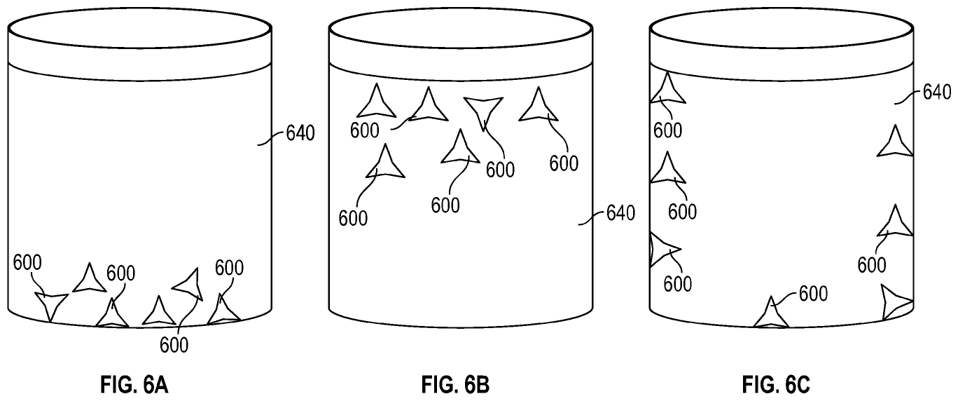

FIGS. 6A-6C depicts STAR particles that have dehomogenized within the vehicle. In FIG. 6A, the STAR particles 600 have settled out of a vehicle at or near the bottom of a storage container 640. In FIG. 6B, the STAR particles 600 float at or near the top of the storage container 640. In FIG. 6C, the STAR particles 600 are adhered to the walls of the storage container 640. STAR particles may settle to the bottom of a storage container when the average density of the STAR particles is greater than the average density of the vehicle being displaced by the STAR particle or when force exerted by the STAR particle on the vehicle exceeds the force that that the vehicle exerts on the STAR particle (i.e., net buoyancy force). The STAR particles may float at the top of the storage container when the average density of the STAR particles is less than the average density of the vehicle being displaced by the STAR particle. For example, the net buoyancy force may be altered by increasing the average density of the vehicle. Alternatively or additionally, the STAR particle average density may be decreased.

As used herein, the term “density” refers to mass per volume, and “average density” refers to a mean value of density.

The STAR particles may be porous. In a preferred embodiment, the STAR particles are composed of ceramic materials (e.g., alumina, titania, zinc oxide, magnesium oxide) which in their sintered state form a microscopic porous ceramic structure. Similarly, poly meric STAR particle can be produced that form a microscopic porous structure. The STAR particle average density may be altered by increasing the porosity of the STAR particle such that air or gas pockets remain trapped inside the STAR particle. Changing the STAR particle porosity may be accomplished by changing ceramic or polymer particle size, sintering or heating temperature, processing conditions, and/or ceramic or polymer formulation. The STAR particle average density may also be altered by modifying the materials used to fabricate the STAR particles. The STAR particle net buoyancy force may also be modified by changing the geometry or size of the STAR particle. For example, a larger STAR particle will displace more vehicle volume, and a smaller STAR particle will displace less vehicle volume, thereby altering the net buoyancy force.

STAR particles that are adhered to walls of a storage container may be more likely to agglomerate. Concentration in a particular space, or an uneven distribution of STAR particles within the storage container often may be undesirable. Rather, it is generally preferred that the STAR particles are roughly evenly dispersed within the vehicle.

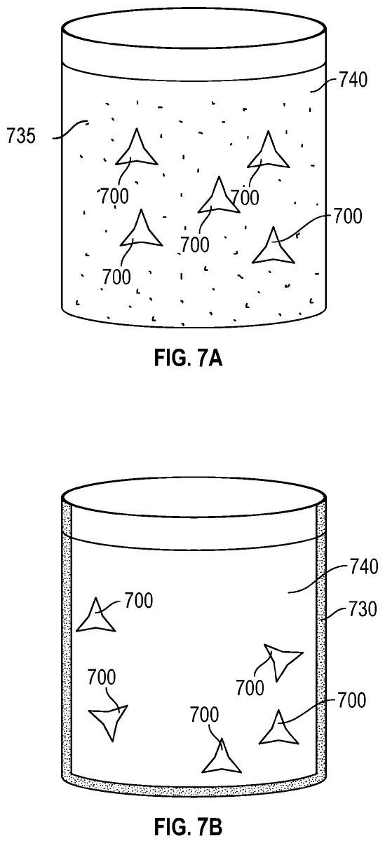

A variety of techniques may be employed for retarding dehomogenization of the STAR particles, to help maintain an even dispersion of STAR particles within a vehicle, for example within a storage container. In some embodiments, the vehicle composition is adjusted to match the density of the STAR particle density, and/or a suitable dispersant or other additive is included in the vehicle to mitigate or prevent the STAR particles from settling or floating or adhering on the container walls. In FIG. 7A, an additive 735 is included in the vehicle so that the STAR particles 700 remain evenly dispersed within the vehicle. In FIG. 7B, the walls of the storage container 740 are coated in a material 730 to prevent adhesion of the STAR particles to the inner walls of the storage container 740.

The compositions of STAR particles dispersed in a fluid vehicle preferably maintain a substantially homogenous dispersion even when the average densities of the STAR particles and the vehicle are different from one another. In this context, the term “maintain” means for durations that are sufficiently long to be practical after manufacture, including shipping and storage prior to use by end users. For instance, the durations may range from a few weeks to one or more years, for example from 2 weeks to 48 weeks.

### EMBODIMENTS

Some embodiments of the present disclosure can be described in view of one or more of the following:

Embodiment 1. A packaged STAR particle composition comprising a plurality of STAR particles which are configured to mechanically disrupt a biological tissue, a vehicle in which the plurality of STAR particles are dispersed, and a container comprising the vehicle and STAR particles, wherein the STAR particles, the vehicle, and the container are configured to maintain a substantially homogenous dispersion of the STAR particles within the vehicle and within the container.

Embodiment 2. The packaged STAR particle composition of Embodiment 1, wherein the STAR particles and the vehicle have similar average densities.

Embodiment 3. The packaged STAR particle composition of either of Embodiments 1 or 2, wherein the vehicle has a viscosity effective to prevent or limit dehomogenization of the STAR particles even when the average densities of the STAR particles and the vehicle are different from one another.

Embodiment 4. The packaged STAR particle composition of any one of Embodiments 1 to 3, wherein the vehicle comprises at least one dispersant.

Embodiment 5. The packaged STAR particle composition of any one of Embodiments 1 to 4, wherein the container walls in contact with the vehicle have surface properties that inhibit adherence of the STAR particles thereto, optionally for example wherein the container walls have a coating effective to inhibit adherence of the STAR particles thereto.

Embodiment 6. The packaged STAR particle composition of any one of Embodiments 1 to 5, wherein the STAR particles possess electrostatic, steric hindrance, and/or magnetic properties that generate repulsive forces between the STAR particles, effective to prevent or limit agglomeration of the STAR particles.

Embodiment 7. The packaged STAR particle composition of any one of Embodiments 1 to 6, further comprising at least one salt compound adapted to reduce the Debye length of a charge on the STAR particles.

Embodiment 8. The packaged STAR particle composition of any one of Embodiments 1 to 7, further comprising a surfactant adapted to inhibit interactions between hydrophobic or hydrophilic surfaces on the STAR particles.

Embodiment 9. A composition comprising a plurality of STAR particles, and a vehicle in which the plurality of STAR particles are dispersed, wherein the vehicle has a viscosity of at least 100 cP, or greater than 100 cP, such that the viscosity of the vehicle is effective to at least partly maintain the dispersion of the STAR particles within the vehicle and/or limit dehomogenization. Embodiment 10. A composition comprising a plurality of STAR particles having a structure formed at least partially of a first material, and a vehicle in which the plurality of STAR particles are dispersed, wherein the vehicle comprises a solvent for the first material, wherein the composition is configured such that the plurality of STAR particles are resistant to dissolution and/or unintended deactivation within the vehicle.

Embodiment 11. The composition of either of Embodiments 9 or 10, wherein the STAR particles have a coating thereon which is substantially insoluble in the solvent of the vehicle.

Embodiment 12. The composition of any one of Embodiments 9 to 11, wherein the STAR particles are encapsulated in an encapsulation material which is substantially insoluble in the solvent of the vehicle.

Embodiment 13. The composition of any one of Embodiments 9 to 12, wherein the vehicle is saturated with solute effective to prevent or limit dissolution of the first material in the solvent of the vehicle.

Embodiment 14. The composition of any one of Embodiments 9 to 13, wherein the structure of the STAR particle is further formed of a second material which is substantially insoluble in the solvent of the vehicle.

Embodiment 15. A composition comprising a plurality of STAR particles, and a vehicle in which the plurality of STAR particles are dispersed, wherein the STAR particles are adapted to degrade following application and use (i) by a selected change in pH, osmolarity, temperature, or ionic composition of the vehicle, or (ii) in response to an external stimulus.

Embodiment 16. The composition of Embodiment 15, wherein the external stimulus comprises exposure to atmospheric oxygen, light, or water.

Embodiment 17. A packaged STAR particle composition comprising a plurality of STAR particles which are configured to mechanically disrupt a biological tissue, a vehicle in which the plurality of STAR particles are dispersed, and a container comprising the vehicle and STAR particles, wherein the STAR particles and/or walls of the container in contact with the vehicle are coated with a cushioning material which is more deformable than the materials forming the STAR particles and/or the walls of the container.

Embodiment 18. A composition comprising a plurality of STAR particles which are configured to mechanically disrupt a biological tissue, wherein the STAR particles having a surface coating composed of a material that is mechanically stronger than the underlying material(s) forming the STAR particles.

Embodiment 19. The composition of Embodiment 18, wherein the underlying material(s) comprise an organic material and the surface coating material comprises an inorganic material.

Embodiment 20. The composition of any one of Embodiments 1 to 19, wherein the composition comprises a substance of interest (SOI).

Embodiment 21. The composition of any one of Embodiments 1 to 20, wherein the SOI comprises a bioactive agent.

Embodiment 22. The composition of any one of Embodiments 1 to 21, wherein the SOI is located in and/or on the STAR particles.

Embodiment 23. The composition of any one of Embodiments 1 to 22, wherein the SOI is located in the vehicle.

Embodiment 24. A method of administering a substance of interest (SOI) to a patient's skin, the method comprising applying the composition of any one of Embodiments 20 to 23 onto a skin surface, and manipulating the composition to cause the STAR particles to mechanically disrupt the skin surface.

Embodiment 25. A STAR particle composition comprising a plurality of STAR particles which are configured to mechanically disrupt a biological tissue, a plurality of SOI particles separate from the plurality of STAR particles, and a liquid vehicle in which the plurality of STAR particles and the plurality of SOI particles are dispersed.

Embodiment 26. The composition of Embodiment 25, wherein the STAR particles and/or the SOI particles have a coating thereon effective to substantially prevent dissolution of the STAR particles and/or the SOI particles in the liquid vehicle.

Embodiment 27. A STAR particle composition comprising a plurality of STAR particles which are configured to mechanically disrupt a biological tissue, a first liquid phase, and a second liquid phase, wherein the plurality of STAR particles are dispersed in the first or second liquid phase, and a substance of interest (SOI) is provided in the first or second liquid phase.

Embodiment 28. The composition of Embodiment 27, wherein first liquid phase is a continuous phase and the second liquid phase is a discontinuous phase dispersed in the first liquid phase.

Embodiment 29. The composition of either of Embodiments 27 or 28, wherein the STAR particles are dispersed in only one of the first liquid phase or the second liquid phase, and the SOI is dissolved in the other of the first liquid phase or the second liquid phase.

Embodiment 30. The composition of any one of Embodiments 27 to 29, wherein (i) the STAR particles are dispersed only in the first liquid phase, and the SOI is dissolved (or otherwise provided) in the first liquid phase and the second liquid phase; (ii) the STAR particles are dispersed in the first liquid phase and the second liquid phase, and the SOI is dissolved (or otherwise provided) only in the first liquid phase; (iii) the SOI is dissolved (or otherwise provided) in the first liquid phase and the second liquid phase, and the STAR particles are dispersed only in the second liquid phase; (iv) the STAR particles are dispersed in the first liquid phase and the second liquid phase, and the SOI is dissolved (or otherwise provided) only in the second liquid phase; (v) the STAR particles are dispersed only in the first liquid phase, and the SOI is dissolved (or otherwise provided) only in the second liquid phase; (vi) the SOI is dissolved (or otherwise provided) only in the first liquid phase, and the STAR particles are dispersed only in the second liquid phase; or (vii) the STAR particles are dispersed in the first liquid phase and the second liquid phase, and the SOI is dissolved (or otherwise provided) in the first liquid phase and the second liquid phase.

As used in this specification and the appended claims, the singular forms “a,” “an,” and “the” include plural referents unless the content clearly dictates otherwise. The term “about”, as used herein, indicates the value of a given quantity can include quantities ranging within 10% of the stated value, or optionally within 5% of the value, or in some embodiments within 1% of the value.

Modifications and variations of the methods and systems described herein will be obvious to those skilled in the art from the foregoing detailed description. Such modifications and variations are intended to come within the scope of the appended claims.

1. A packaged STAR particle composition comprising:
a plurality of STAR particles which are configured to mechanically disrupt a biological tissue;
a vehicle in which the plurality of STAR particles are dispersed; and
a container comprising the vehicle and STAR particles,
wherein the STAR particles, the vehicle, and the container are configured to maintain a substantially homogenous dispersion of the STAR particles within the vehicle and within the container.

2. The packaged STAR particle composition of claim 1, wherein the STAR particles and the vehicle have similar average densities.

3. The packaged STAR particle composition of claim 1, wherein the vehicle has a viscosity effective to prevent or limit dehomogenization of the STAR particles even when the average densities of the STAR particles and the vehicle are different from one another.

4. The packaged STAR particle composition of claim 1, wherein the vehicle comprises at least one dispersant.

5. The packaged STAR particle composition of claim 1, wherein the container walls in contact with the vehicle have surface properties that inhibit adherence of the STAR particles thereto, optionally for example wherein the container walls have a coating effective to inhibit adherence of the STAR particles thereto.

6. The packaged STAR particle composition of claim 1, wherein the STAR particles possess electrostatic, steric hindrance, and/or magnetic properties that generate repulsive forces between the STAR particles, effective to prevent or limit agglomeration of the STAR particles.

7. The packaged STAR particle composition of claim 6, further comprising at least one salt compound adapted to reduce the Debye length of a charge on the STAR particles.

8. The packaged STAR particle composition of claim 6, further comprising a surfactant adapted to inhibit interactions between hydrophobic or hydrophilic surfaces on the STAR particles.

9. A composition comprising:
a plurality of STAR particles; and
a vehicle in which the plurality of STAR particles are dispersed,
wherein the vehicle has a viscosity of at least 100 cP, such that the viscosity of the vehicle is effective to at least partly maintain the dispersion of the STAR particles within the vehicle and/or limit dehomogenization.

10. A composition comprising:
a plurality of STAR particles having a structure formed at least partially of a first material; and
a vehicle in which the plurality of STAR particles are dispersed, wherein the vehicle comprises a solvent for the first material,
wherein the composition is configured such that the plurality of STAR particles are resistant to dissolution and/or unintended deactivation within the vehicle.

11. The composition of claim 10, wherein the STAR particles have a coating thereon which is substantially insoluble in the solvent of the vehicle.

12. The composition of claim 10, wherein the STAR particles are encapsulated in an encapsulation material which is substantially insoluble in the solvent of the vehicle.

13. The composition of claim 10, wherein the vehicle is saturated with solute effective to prevent or limit dissolution of the first material in the solvent of the vehicle.

14. The composition of claim 10, wherein the structure of the STAR particle is further formed of a second material which is substantially insoluble in the solvent of the vehicle.

15-17. (canceled)

18. A composition comprising:
a plurality of STAR particles which are configured to mechanically disrupt a biological tissue,
wherein the STAR particles having a surface coating composed of a material that is mechanically stronger than the underlying material(s) forming the STAR particles.

19. The composition of claim 18, wherein the underlying material(s) comprise an organic material and the surface coating material comprises an inorganic material.

20. The composition of claim 1, wherein the composition comprises a substance of interest (SOI).

21. The composition of claim 20, wherein the SOI comprises a bioactive agent.

22. The composition of claim 20, wherein the SOI is located in and/or on the STAR particles.

23. The composition of claim 20, wherein the SOI is located in the vehicle.

24. A method of administering a substance of interest (SOI) to a patient's skin, the method comprising:
applying the composition of claim 20 onto a skin surface; and
manipulating the composition to cause the STAR particles to mechanically disrupt the skin surface.

25. A STAR particle composition comprising:
a plurality of STAR particles which are configured to mechanically disrupt a biological tissue;
a plurality of SOI particles separate from the plurality of STAR particles; and
a liquid vehicle in which the plurality of STAR particles and the plurality of SOI particles are dispersed.

26. The composition of claim 25, wherein the STAR particles and/or the SOI particles have a coating thereon effective to substantially prevent dissolution of the STAR particles and/or the SOI particles in the liquid vehicle.

27-31. (canceled)
